(19) United States
(12) Patent Application Publication
(10) Pub. No.: US 2006/0211966 A1
(43) Pub. Date: Sep. 21, 2006

(54) Knee joint for orthosis

(76) Inventors: Dale L. Hatton, Mount Ida, AR (US); Zane G. Wallace, Story, AR (US); Gary W. Horton, Little Rock, AR (US)

Correspondence Address: J. CHARLES DOUGHERTY, 200 WEST CAPITOL AVE, SUITE 2300, LITTLE ROCK, AR, 72201, US

(21) Appl. No.: 11/374,258

(22) Filed: Mar. 13, 2006

Related U.S. Application Data
(60) Provisional application No. 60/661,995, filed on Mar. 14, 2005.

(51) Int. Cl. A61F 5/00

(52) U.S. Cl. 602/16

(57) ABSTRACT

An orthosis knee joint using a lock bearing arrangement is disclosed. The joint uses lock bearings that move in individual, arcuate races. The lock bearings are manipulated by means of a bearing cage from a locked position to an unlocked position. The locking mechanism is provided by the ramped shape of the bottom of the individual bearing races; movement of the bearing cage pinches the bearings at the shallower end of the ramp when the lock is activated and rotational force is exerted in the flexion direction. Movement in the extension direction, however, is still allowed in the locked position.

### BACKGROUND OF THE INVENTION

This application claims benefit of U.S. provisional patent application Ser. No. 60/661,995, entitled “Knee Joint for Orthosis” and filed on Mar. 14, 2005. The entire disclosure of such provisional patent application is incorporated herein by reference.

The present invention relates to knee joints for orthoses, and in particular to knee joints that are capable of selectively locking and unlocking in response to user motion.

For many years, orthopedic appliances have been used to provide support and control for a patient who has temporarily or permanently lost muscular or neurological control of a limb. Leg orthoses (or braces) designed for this purpose must have some means to allow the leg to bend at the knee. Leg braces thus typically consist of three. principal components: a structure rigidly attached to the upper leg, a structure rigidly attached to the lower leg, and a joint or joints at the knee connecting the upper and lower leg structures.

In order to support the patient while walking, the knee joint of a leg orthosis must be capable of locking; otherwise the brace would not support the patient as weight in placed on the leg. The simplest designs for such devices comprise a manual locking feature. In these devices, the brace is locked whenever the patient is standing or walking, and then may be unlocked to allow the patient to comfortably sit. Such designs do not allow for a natural movement of the leg, however, since the knee cannot bend during walking.

During normal walking, the knee undergoes a complex flexion and extension range of motions. In order to approximate this movement with a leg orthosis, the knee joint of the orthosis must selectively lock and unlock while the patient is in motion. Ideally, the knee joint would remain unlocked when no weight is placed on the leg, but lock with respect to its flexion movement when the patient places weight on the leg for support. Thus the leg would be able to undergo a more natural flexion and then extension motion as weight is placed on the opposite leg, allowing the leg bearing the brace to be brought forward in a natural sweeping movement.

A number of attempts have been made to develop orthosis knee joints that allows a more natural knee movement as just described. U.S. Pat. No. 6,960,175 to Myers, for example, discloses a joint locking mechanism that comprises a ratchet ring with a pair of internal cams. The ratchet ring and cams feature interlocking teeth, and the cams are spring loaded such that they allow rotation of the ratchet ring in the knee extension direction while bearing a load, but prevent flexion rotation. An actuator rod controls the locking and unlocking mechanism; this rod is positioned such that it contacts the ground when weight is applied to the braced leg, thereby activating the locking mechanism when weight is placed on the leg.

U.S. Pat. No. 6,770,045 to Naft teaches an orthosis knee joint that is controlled by an electronic sensor in conjunction with an electronic circuit. The joint locking mechanism is provided by opposing ratchet plates having radially cut teeth. Due to the manner in which the teeth are cut, the joint allows movement in the extension direction but does not allow movement in the flexion direction when the plates are in contact.

U.S. Pat. No. 6,139,586 to Wagner et al. teaches a knee joint for a leg prosthesis that utilizes needle rollers for a braking or locking action. The needle rollers surround the joint shaft, and an indexing wedge is positioned to extend into the needle roller race when pressure is applied to the upper portion of the prosthesis. In this manner, weight on the upper part of the prosthesis causes the joint to brake or lock with respect to flexion motion, while releasing the weight on the prosthesis will cause the joint to unlock.

U.S. Pat. No. 4,451,939 to Thompson teaches a knee joint for a leg prosthesis with a roller bearing lock or clutch assembly. The lock mechanism comprises a race within a clutch assembly with a cammed inner surface. An operator member fits over the face of the clutch assembly and holds the bearings in a certain orientation with respect to the cammed inner surface. A notch is provided in the operator member whereby it may be rotated, such that movement of the operator member moves the bearings into a relationship with the cammed inner surface that locks or unlocks the mechanism with respect to rotation in the flexion direction. An actuator arm is in communication with the notch in the operator member, with the actuator arm being moved in response to a spring-loaded button in the foot of the prosthesis. Pressure on the prosthesis thereby rotates the operator member, which in turn moves the bearings into the lock position.

U.S. Pat. No. 5,899,869 to Barrack, Jr. et al. teaches a leg orthosis knee joint with roller bearings that fit between a notched brake hub and a split brake band means. The splits in the brake band allow it to be pressed outward and thereby radially expanded. A switch, which activates in response to pressure from the lower section of the orthosis, moves the hub such that the bearings push out on the split brake band means, forcing its segments into a brake drum circumscribing the split brake band means, and thereby preventing rotation of the joint in the flexion direction.

Each of the devices described above function, to a greater or lesser degree, to provide support for a patient during walking by preventing flexion at the knee when weight is applied to the leg but allowing free rotation when weight is not applied. It is desirable, however, to reduce the size and weight of a knee joint orthosis to the greatest degree possible in order to create an orthosis that is more comfortable for the patient to wear. In addition, since orthosis knee joints are subject to significant forces and repetitive use, durability is a critical consideration in any such design. Finally, safety is a paramount concern, since the patient must be assured that when weight is applied to the device that the lock will not fail; otherwise, serious injury could result to the patient as the result of a fall.

It would therefore be desirable to develop an improved orthosis knee joint that is selectively lockable but is simple to construct, relatively lightweight, durable, and dependable.

### SUMMARY OF THE INVENTION

The present invention is directed to an orthosis knee joint using a lock bearing arrangement whereby the lock bearings move in individual, arcuate races. The lock bearings are manipulated by means of a bearing cage from a locked position to an unlocked position. The locking mechanism is provided by the ramped shape of the bottom of the individual bearing races; movement of the bearing cage pinches the bearings at the shallower end of the ramp when the lock is activated and rotational force is exerted in the flexion direction. Movement in the extension direction, however, is still allowed in the locked position. Activation of the locking mechanism may be by a filament or rod attached to the bearing cage, which may be manipulated, for example, by means of a pressure switch at the base of the orthosis. The joint may thus be selectively locked and unlocked in response to weight being placed on the leg to which the orthosis is fitted.

It is therefore an object of the present invention to provide for a knee joint for an orthosis that prevents flexion at the joint when locked and allows free rotation at the joint when unlocked.

It is a further object of the present invention to provide for a knee joint orthosis that locks in response to weight being applied to the leg to which the orthosis is fitted.

It is also an object of the present invention to provide for a knee joint orthosis that is mechanically simple to construct and durable.

It is also an object of the present invention to provide for a knee joint orthosis that is relatively small and lightweight to increase the comfort of wearing the orthosis for the patient.

These and other features, objects and advantages of the present invention will become better understood from a consideration of the following detailed description of the preferred embodiments and appended claims in conjunction with the drawings as described following:

### DRAWINGS

FIG. 1 is a perspective, exploded view of a preferred embodiment of the present invention.

FIG. 2 is an end-on elevational view of a preferred embodiment of the present invention.

FIG. 3A is a side elevational view, in partial cut-away, of a preferred embodiment of the present invention in the locked position.

FIG. 3B is a detail view of a lock bearing of a preferred embodiment of the present invention in the locked position.

FIG. 4A is a side elevational view, in partial cut-away, of a preferred embodiment of the present invention in the unlocked (or “home”) position.

FIG. 4B is a detail view of a lock bearing of a preferred embodiment of the present invention in the unlocked (or “home”) position.

### DETAILED DESCRIPTION OF THE PREFERRED EMBODIMENTS

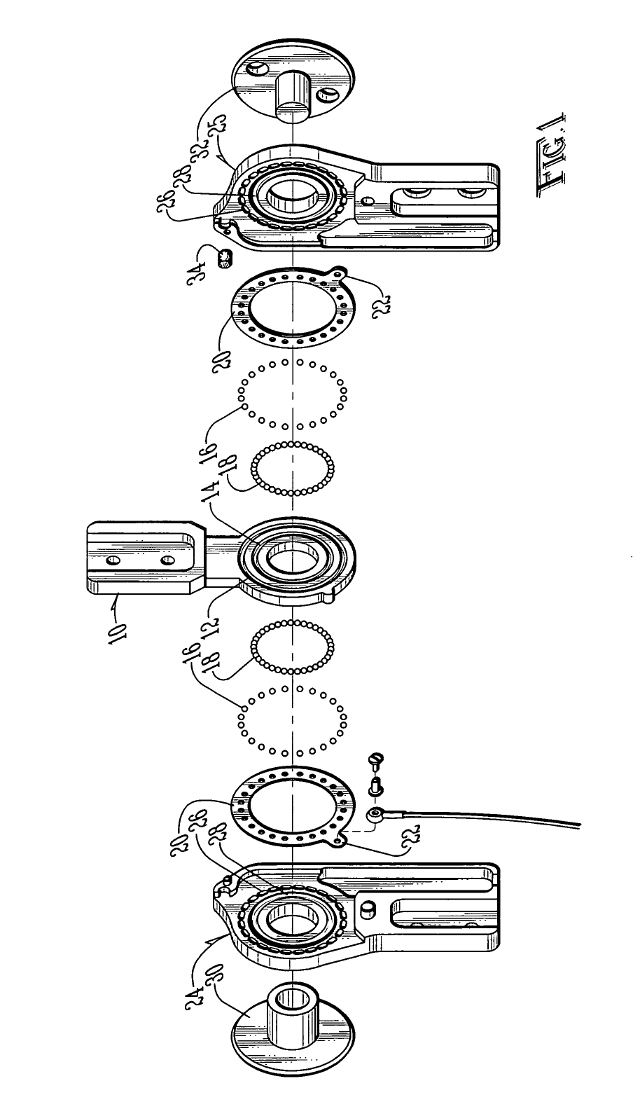

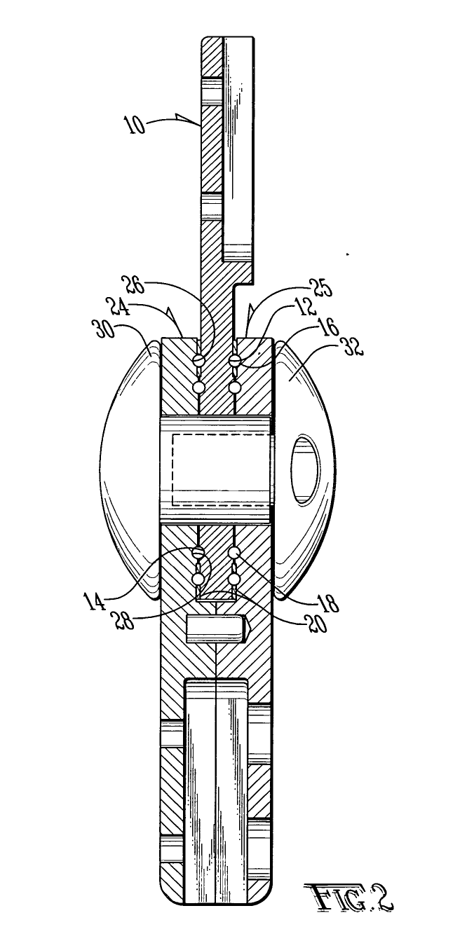

With reference to FIGS. 1 and 2, the principal components of a preferred embodiment of the present invention may now be described. Upper arm 10 is configured to be attached to the upper rigid portion of an orthosis (not shown) that is fitted to a patient's upper leg. On the face of each side of upper arm 10 is an upper thrust bearing race 14. Circumscribing upper thrust bearing race 14 is upper lock bearing race 12. Both upper thrust bearing races 14 and upper lock bearing races 12 are annular depressions in the face of upper arm 10. Preferably, upper arm 10 is formed of steel or a similarly strong material, and upper thrust bearing races 14 and upper lock bearing races 12 are machined into the faces of upper arm 10.

Resting in upper thrust bearing races 14 and upper lock bearing races 12 are thrust bearings 18 and lock bearings 16, respectively. Thrust bearings 18 provide for smooth rotation of upper arm 10 with respect to lower arms 24 and 25. Lock bearings 16 provide the locking mechanism of the joint, as will be described more fully below. Thrust bearings 18 and lock bearings 16 are preferably formed of steel for strength and for the ability of the material to resist deformation. A bearing cage 20 is fitted at each side of upper arm 10 adjacent to upper lock bearing race 12. Because each bearing cage 20 features an opening sized to fit each of lock bearings 16, each bearing cage 20 holds each lock bearing 16 in a set position with respect to bearing cage 20.

On either side of upper arm 10 and the associated bearing cage 20 is fitted one of lower arms 24 and 25. In the preferred embodiment, lower arms 24 and 25 are designed as mirror images of each other, such that when assembled as shown in FIG. 2 they fit together to form a smooth silhouette. Similar to upper arm 10, lower arms 24 and 25 are intended to be attached to the lower rigid portion of an orthosis (not shown) that is fitted to a patient's lower leg. On the interior face of each of lower arms 24 and 25 is a lower thrust bearing race 28. Circumscribing each lower thrust bearing race 28 is a plurality of arcuate lower lock bearing races 26. Lower thrust bearing races 28 are annular depressions in the face of lower arms 24 and 25, aligned with and symmetrical to upper thrust bearing races 14. Lower lock bearing races 26, however, are formed in an series of short races together forming an interrupted annular depression, with each of lock bearing races 26 matching the location of a particular lock bearing 16 and associated opening on bearing cage 20. It will be seen that during assembly lock bearings 16, and correspondingly bearing cage 20, must be properly aligned with the lower lock bearing races 26 for the upper arm 10 and lower arms 24 and 25 to properly be fit together. As with upper arm 10, lower arms 24 and 25 are preferably formed of steel or a similarly strong material, and lower thrust bearing races 14 and upper lock bearing races 12 are machined into the faces of upper arm 10.

Nut 30 and bolt 32 pass through the opening in upper arm 10 and lower arms 24 and 25, fastening together to hold the various components together while allowing rotation between upper arm 10 and lower arms 24 and 25 around the shaft of nut 30. In the preferred embodiment, nut 30 and bolt 32 fit together by means of a press fit, but threads or other means of attaching nut 30 to bolt 32 may be employed, so long as free rotation of upper arm 10 with respect to lower arms 24 and 25 when the joint is unlocked is maintained.

In the preferred embodiment, rotation of upper arm 10 with respect to lower arms 24 and 25 should be limited even in the unlocked position such that the rotation does not exceed that which would be encountered with a human knee during normal walking movements. Stops 34 extending from lower arms 24 and 25 are employed in the preferred embodiment for this purpose. Stops 34 extend towards each other and block progress of upper arm 10 past the point where it contacts stops 34. In the preferred embodiment, total extension of the joint is limited to no more than 130 degrees.

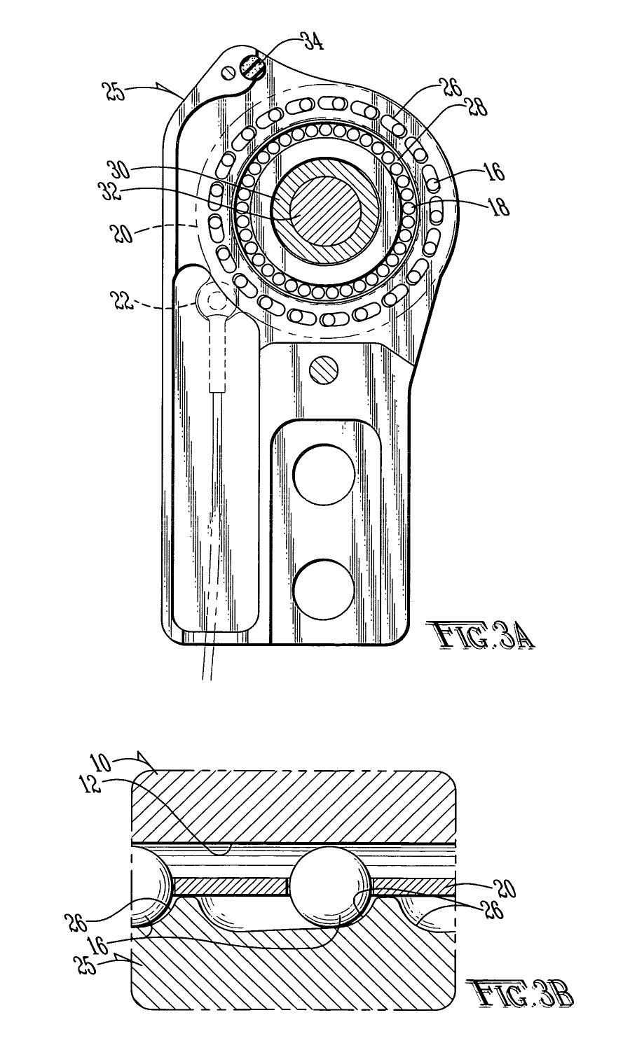

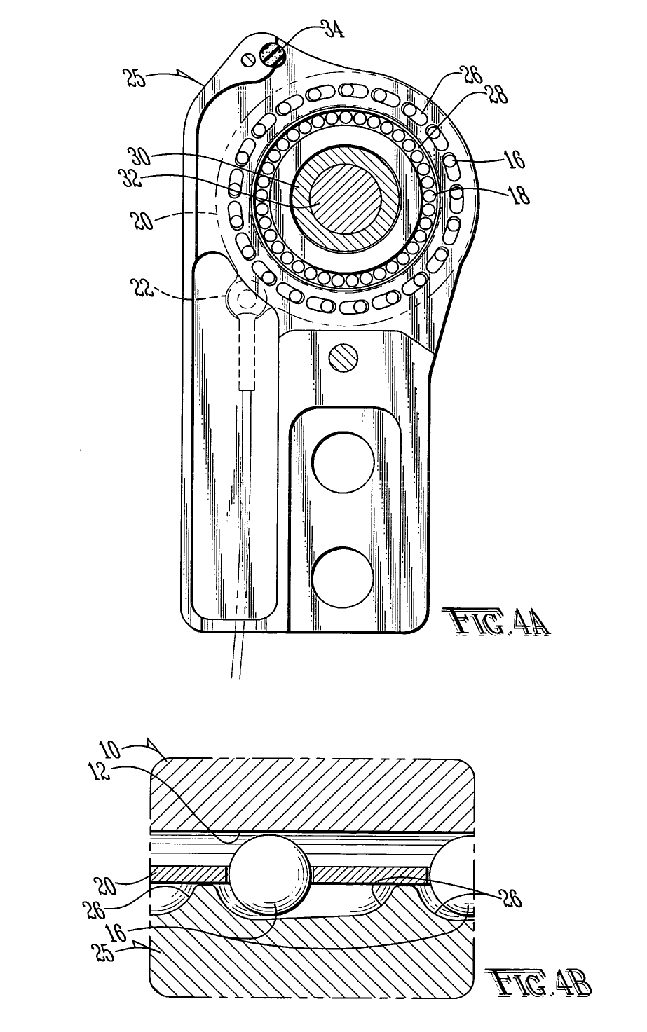

Referring now to FIGS. 3A, 3B, 4A, and 4B, the operation of the preferred embodiment of the present invention may now be described. In FIGS. 3A and 3B, the preferred embodiment is depicted in the locked condition. Each lower lock bearing race 26 is ramped at its inner surface as depicted in FIG. 3B. By rotational movement of bearing cage 20, each lock bearing 16 is forced into the shallower end of the corresponding lower lock bearing race 26. When rotation of the joint is attempted in the flexion direction in this condition, the bearing is “pinched” into this shallow region, and due to friction the rotation of upper arm 10 with respect to lower arms 25 and 26 is prevented. FIGS. 4A and 4B depict the preferred embodiment in the “home” or unlocked position. In this case, bearing cage 20 is rotated slightly in the opposite direction, allowing each lock bearing 16 to remain in the deeper portion of the corresponding lower lock bearing race 26 when flexion rotation is attempted. The joint is thus allowed to rotate freely in both the flexion and extension directions when in this state.

The rotation of bearing cages 20 which allows the locking and unlocking of the invention may be performed by means of a flexible filament, activator rod, or other activation means as are known in the art. Tab 22 may be used to provide the mechanical linkage between bearing cages 20 and the activation means employed in any particular embodiment. In the preferred embodiment, a monofilament line (not shown) is employed for this purpose due to its strength and flexibility. A sheath (also not shown) may optionally be employed around the monofilament line between bearing cage 20 and a switch or other activation means used to sense weight being placed upon the leg bearing the orthosis and thereby activate the locking mechanism. In alternative embodiments, the locking and unlocking of the invention may be controlled by means of an electric or electronic signal. An electronic controller may be used in connection with the invention for the purpose of sending an appropriate signal in response to a control stimulus, such as the activation of an electrical or electronic sensor. In certain of these alternative embodiments, a solenoid may be used to activate the locking or unlocking mechanism in response to an electrical signal from the controller.

It may be noted that the preferred embodiment has been described with certain features associated with an upper arm and certain features associated with a two-piece lower arm of an orthosis. These features could easily be flipped to the opposite arms in an alternative embodiment of the device. Likewise, the upper arm could be formed of two pieces while the lower arm is a single piece in an alternative embodiment. While the term “arm” is used throughout to describe the main components of the preferred embodiment that act as a housing for the bearing components and connect with the orthosis upper and lower leg fittings, it should be understood that the “arm” components are not restricted to an elongated shape, and may in alternative embodiments comprise any shape capable of fulfilling the functions described herein.

It may further be noted that while the invention has been described for use in connection with an orthosis, the device could also be used with a prosthesis or other devices intended to aid a person in walking. In addition, the invention might in alternative embodiments be employed with respect to spacesuits, pressure suits, robots, or other applications where a knee joint is indicated and a more natural or human-like gait is desired.

The present invention has been described with reference to certain preferred and alternative embodiments that are intended to be exemplary only and not limiting to the full scope of the present invention as set forth in the appended claims.

We claim:

1. A selectively locking joint, comprising:
(a) a first arm comprising an arcuate bearing race at a first face;
(b) a lock bearing fitted into said bearing race;
(c) a bearing cage fitted at said first face and circumscribing said bearing; and
(d) a second arm fitted opposite said first arm at said first face with said bearing cage therebetween and selectively rotatable with respect to said first arm.

2. The joint of claim 1, wherein said arcuate bearing race has a ramped lower surface such that said bearing race comprises a shallow end and a deep end.

3. The joint of claim 2, wherein said second arm comprises a bearing race opposite said first arm bearing race and adapted to receive said lock bearing.

4. The joint of claim 3, wherein said bearing cage is rotatable with respect to said first arm between a locked position and an unlocked position.

5. The joint of claim 4, wherein said bearing cage is positioned in said locked position such that said lock bearing is pinched against said arcuate bearing race shallow end when rotational force is applied between said first arm and said second arm in a flexion direction.

6. The joint of claim 5, wherein said bearing cage is positioned in said unlocked position such that said lock bearing is free to rotate at said arcuate bearing race deep end.

7. The joint of claim 6, wherein said bearing cage comprises a tab adapted to receive a control means for manipulation of said bearing cage between said locked and said unlocked position.

8. The joint of claim 1, wherein said first arm comprises a plurality of arcuate bearing races at said first face, and said joint further comprises a plurality of lock bearings, wherein each of said lock bearings is fitted into one of said plurality of arcuate bearing races, and said bearing cage comprises a plurality of openings, wherein each of said openings is circumscribing one of said plurality of lock bearings.

9. The joint of claim 8, wherein each of said arcuate bearing races lie along a circular region at said first arm first face.

10. The joint of claim 1, wherein said first arm comprises an arcuate bearing race at a second face; a lock bearing fitted into said bearing race at said second face; a second bearing cage fitted at said second face and circumscribing said bearing at said second face; and a third arm fitted opposite said first arm at said second face with said second bearing cage therebetween and selectively rotatable with respect to said first arm in conjunction with said second arm.
